(19) United States
(12) Patent Application Publication
(10) Pub. No.: US 2025/0347392 A1
(43) Pub. Date: Nov. 13, 2025

(54) Combined Lamp and Air Purifying System and Method of Use

(71) Applicant: Michael Staggs, Elsinore, UT (US)

(72) Inventor: Michael Staggs, Elsinore, UT (US)

(21) Appl. No.: 19/198,195

(22) Filed: May 5, 2025

Related U.S. Application Data
(60) Provisional application No. 63/643,517, filed on May 7, 2024.

(51) Int. Cl. F21K 9/237; A61L 9/20; F21K 9/235; F21V 23/04; F21V 23/06; F21Y 105/10; F21Y 113/00; F21Y 115/10

(52) U.S. Cl. CPC F21K 9/237; A61L 9/20; F21K 9/235; F21V 23/04; F21V 23/06; A61L 2209/12; A61L 2209/14; A61L 2209/15; F21Y 2105/10; F21Y 2113/30; F21Y 2115/10

(57) ABSTRACT

A combined lamp and air purifier system designed to remove odors, germs, and bacteria from the air while providing illumination is disclosed. The system includes a shell, a fan unit with air inlets, an air filter, and an illumination source capable of emitting UV-C light for air disinfection. The air filter captures the airborne pollutants and expels the purified air. The system can be powered by various means, including light sockets, lamp fixtures, and vehicle lighter ports. The system features a control button for manual operation, a wireless transceiver for remote control, and may include an air freshener module in vehicle versions. The system is designed for easy installation and maintenance, offering an efficient solution for air purification and lighting in diverse environments.

### CROSS-REFERENCE TO RELATED APPLICATION

The present application claims priority to, and the benefit of, U.S. Provisional Application No. 63/643,517, which was filed on May 7, 2024, and is incorporated herein by reference in its entirety.

### FIELD OF THE INVENTION

The present invention generally relates to the field of air purifying systems. More specifically, the present invention relates to a novel air purifying system designed to remove unwanted fumes, germs, bacteria, and odors from the air. The system resembles a light bulb and features a fan unit and an illumination source. An air filter purifies the air received through the fan unit and is expelled to the surrounding. The system can be powered by a light socket, a wall plug, or by batteries. Accordingly, the present disclosure makes specific reference thereto. Nonetheless, it is to be appreciated that aspects of the present invention are also equally applicable to other like applications, devices, and methods of manufacture.

### BACKGROUND

By way of background, rooms in homes, offices, and other buildings commonly suffer from unwanted odors, as well as airborne germs, bacteria, and other debris. Inhaling polluted or contaminated air can pose serious health risks especially to children and elderly. Individuals commonly use traditional air filters and air purifiers to purify the air, however such devices are less effective and are costly to maintain. Traditional purifiers cannot be kept in all areas of a home which leads to their lack of effectiveness. Many individuals use disinfectant spray; however, such use requires periodic manual effort which can be inconvenient and labor-intensive.

Further, standard air filters and purifiers are usually single-function devices, focusing solely on air purification without offering any additional benefits. Individuals need to use multiple electronic devices such as an air filter for air purification, a lamp for illumination, and more. Also, traditional air purifier devices are not compatible with vehicles which leads to problems to users who commute or drive in their vehicles. Accordingly, there is a growing demand for more multifunctional solutions that not only purify the air but also provide other conveniences, such as illumination and aesthetic appeal.

Therefore, there exists a long felt need in the art for an improved air purifying system. There is also a long felt need in the art for an air purifying system that removes odors, germs, and bacteria from the air while simultaneously illuminating a room or area. Additionally, there is a long felt need in the art for a special air purifying device that can be installed in homes, vehicles, and more. Moreover, there is a long felt need in the art for a uniquely designed air purifying system that uses both an air filter and a UV lamp for disinfecting the air. Further, there is a long felt need in the art for an air purifying device that is easy to use and maintain and can be powered by a light socket, a wall plug, or by batteries. Furthermore, there is a long felt need in the art for a combined illumination and air purifying system that is multifunctional. Finally, there is a long felt need in the art for a uniquely designed portable air purifying system that resembles a light bulb and offers a more convenient replacement for air purifiers compared to standard, currently available air purification products.

The subject matter disclosed and claimed herein, in one embodiment thereof, comprises a combined lamp and air purifier system. The system comprises a fan unit, wherein the fan unit has a plurality of inlets adapted to pull air into the system, an illumination source is adapted to provide illumination and emit UV light for disinfecting surrounding air, at least one air filter is adapted to capture and eliminate airborne pollutants and expel purified air to the surroundings and a base extends downwardly from the bottom end of the shell and is adapted to maintain a stable electrical connection with a socket. The system can be powered by a light socket, a wall plug, or by batteries. The system can be manually operated using a control button or remotely using a handheld electronic device.

In this manner, the combined lamp and air purifier system of the present invention accomplishes all of the forgoing objectives and provides users with a novel air purifying system that efficiently removes odors, germs, and bacteria from the air while also serving as a light source. The air purifying system is designed to enhance indoor air quality by effectively removing unwanted fumes, germs, bacteria, and odors from the air. The system is designed to resemble a light bulb and can be easily screwed into standard light sockets or plugged into vehicle cigarette lighter ports. The system includes a control button for manual activation and deactivation, and enables the options for remote control via a wireless transceiver and smartphone app.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a combined lamp and air purifier system. The system comprises a shell having a top end and a bottom end, the shell includes a grid with a plurality of apertures for expelling purified air, a fan unit has a plurality of inlets, the fan unit is adapted to pull air into the system, an illumination source is adapted to provide illumination and emit UV light for disinfecting surrounding air, at least one air filter disposed within the shell and is adapted to capture and eliminate airborne pollutants and expel purified air through the apertures and a base extends downwardly from the bottom end of the shell and is adapted to maintain a stable electrical connection with a light socket or vehicle lighter port.

In yet another embodiment, a combined lamp and air purifier system is disclosed. The system comprises a shell having a top end and a bottom end, wherein the shell includes a grid with a plurality of apertures and is generally cylindrical in shape, a fan unit is coupled to the top end of the shell, the fan unit has a plurality of inlets for drawing air into the system, an illumination source, the illumination source comprises a UV lamp that emits UV-C light within a wavelength range of 250-300 nm for air disinfection, at least one air filter is disposed within the shell and extends along the grid, the air filter is adapted to capture airborne particles larger than 0.2 microns, and a control button is disposed on the shell for manual activation and deactivation of the system.

In another embodiment, a method of purifying air and providing illumination using a combined lamp and air purifier system is disclosed. The method includes the steps installing the system by threading a base of the system into a compatible socket, activating the system using a control button or a remote device, drawing air into the system through a plurality of inlets in a fan unit, purifying the drawn air by passing it through at least one air filter disposed within a shell of the system, emitting UV-C light from an illumination source connected to the fan unit to disinfect the air, and expelling the purified air through a plurality of apertures in a grid within the shell.

In another aspect, the air filter is a High-Efficiency Particulate Air (HEPA) filter adapted to capture at least 99.8% of airborne particles larger than 0.2 microns.

In yet another aspect, the system includes a wireless transceiver operatively coupled to a controller and enables remote control of the system via a handheld computing device.

In still another embodiment, a combined lamp and air purifier system for vehicles is disclosed. The system includes an air filter to capture and eliminate airborne pollutants and expel purified air, an illumination source adapted to provide illumination and emit UV light for disinfecting surrounding air, an air freshener module for dispensing fragrant air, and a base adapted for insertion into a vehicle's cigarette lighter port for providing electrical power to the system.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of combined lamp and air purifier system of the present invention in accordance with the disclosed structure;

FIG. 2 illustrates a perspective view showing the combined lamp and air purifier system of the present invention being screwed into a light bulb threading on a lamp in accordance with the disclosed structure;

FIG. 3 illustrates an exploded view of the air purifying lamp system of the present invention in accordance with the disclosed structure;

FIG. 4 illustrates a perspective view showing communication of a remote device with the air purifying system of the present invention in accordance with the disclosed structure;

FIG. 5 illustrates a perspective view showing the air purifier system purifying air and eliminating pathogens in accordance with one embodiment of the present invention;

FIG. 6 illustrates a flow chart depicting a process of use of the air purifying system of the present invention in accordance with the disclosed structure; and

FIG. 7 illustrates a perspective view showing another embodiment of the air purifier lamp system of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is exists a long felt need in the art for an improved air purifying system. There is also a long felt need in the art for an air purifying system that removes odors, germs, and bacteria from the air while simultaneously illuminating a room or area. Additionally, there is a long felt need in the art for a special air purifying device that can be installed in homes, vehicles, and more. Moreover, there is a long felt need in the art for a uniquely designed air purifying system that uses both an air filter and UV lamp for disinfecting air. Further, there is a long felt need in the art for an air purifying device that is easy to use and maintain and can be powered by a light socket, a wall plug, a lighter socket/port, or by batteries. Furthermore, there is a long felt need in the art for a combined illumination and air purifying system that is multifunctional. Finally, there is a long felt need in the art for a uniquely designed portable air purifying system that resembles a light bulb and offers a more convenient replacement for air purifiers compared to standard, currently available air purification products.

The present invention, in one exemplary embodiment, is a combined lamp and air purifier system. The system comprises a shell having a top end and a bottom end, wherein the shell includes a grid with a plurality of apertures and is generally cylindrical in shape, a fan unit is coupled to the top end of the shell, the fan unit has a plurality of inlets for drawing air into the system, an illumination source, the illumination source comprises a UV lamp that emits UV-C light within a wavelength range of 250-300 nm for air disinfection, at least one air filter is disposed within the shell and extends along the grid, the air filter is adapted to capture airborne particles larger than 0.2 microns, and a control button is disposed on the shell for manual activation and deactivation of the system.

Reference will now be made in detail to the present preferred embodiments of the invention, examples of which are illustrated in the accompanying drawings. Wherever possible, the same reference numerals are used in the drawings and the description to refer to the same or like parts.

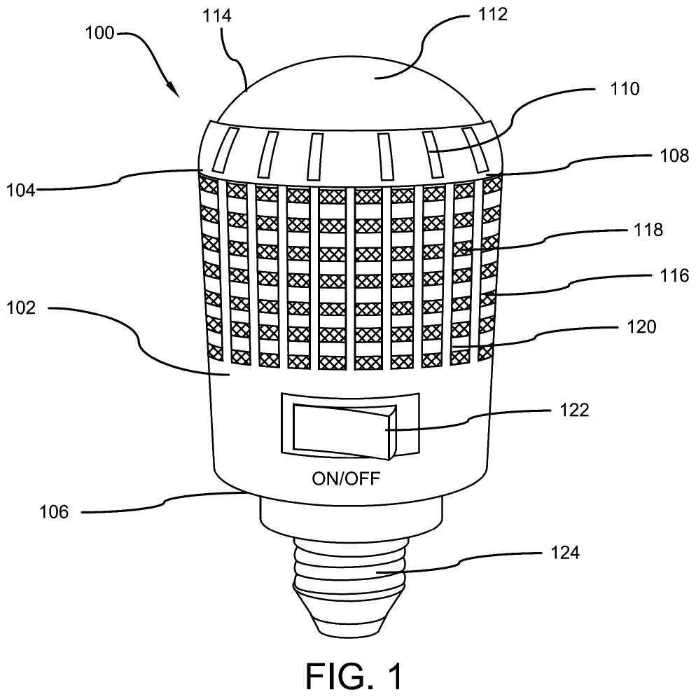

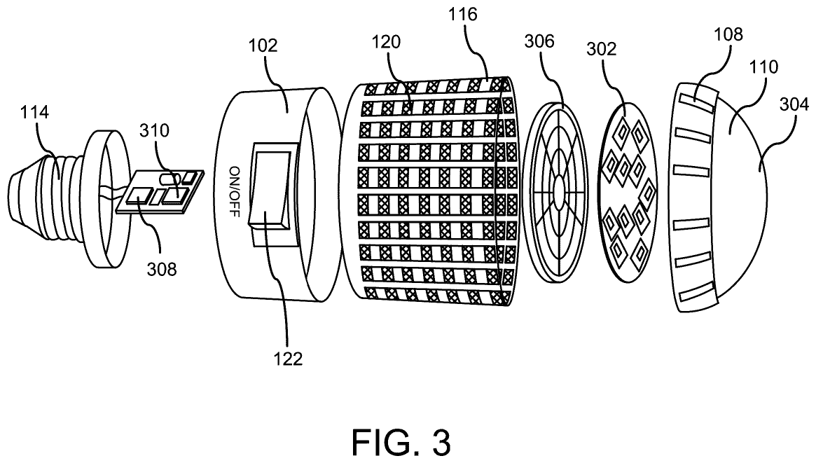

Referring initially to the drawings, FIG. 1 illustrates a perspective view of combined lamp and air purifier system of the present invention in accordance with the disclosed structure. The combined lamp and air purifier system 100 of the present invention is designed as an air purifying system that removes odors, germs, and bacteria from the air and illuminates a room or area. The combined lamp and air purifier system 100 can be designed in different shapes and sizes and can be installed in homes, vehicles, and more to purify air and provide illumination. More specifically, the combined lamp and air purifier system 100 includes a shell 102 having a top end 104 and a bottom end 106. The shell 102 is generally cylindrical but can be of any other geometric shape to meet requirements of different users. A fan unit 108 has a plurality of inlets 110 and is in threaded connection with top end 104 of the shell 102. The fan unit 108 is adapted to pull air from the room or any other area in which the system 100 is placed for purification by the system 100 and is coupled to a fan (illustrated in FIG. 3).

An illumination source 112 such as an LED of a specific color or of multiple colors is disposed on the top end 104 of the shell. The illumination source 112 is preferably convex and extends above the top end 114 of the fan 108. The illumination source 112 is adapted to provide illumination for illuminating a room or any other area. In the preferred embodiment, the illumination source 112 is operatively connected to the top end 114 of the fan unit 108 and can be removed to replace with a new source. In the preferred embodiment, the illumination source 112 is a UV lamp and is used for disinfecting surrounding air. The UV lamp can emit UV-C in a wavelength range of 200-800 nm and preferably, in the range of 250-300 nm. The UV light emitted from the illumination source 112 is safe for human absorption and can kill all types of microorganisms.

The shell 102 includes a grid 116 having a plurality of apertures 118 disposed in the shell 102. The grid 116 forms an area from about 50% to about 80% of the total area of the shell 102 and the apertures 118 enables the purified air to expel from the system 100. At least one air filter 120 is disposed in the system 100 for purifying the air received from the fan inlets 110. The purified air is directly expelled from the air filter 120 through the plurality of apertures 118 for circulating purified air in the surroundings. In the preferred embodiment, the air filter 120 extends along the grid 116 for providing purified air in a 360-degree area. The air filter 120 is adapted to capture and eliminate pollutants from the air and can be one or more of High-Efficiency Particulate Matter (HEPA) filter, activated carbon filter, ionic filter, and electrostatic precipitator. Preferably, the filter 120 is adapted to capture at least 99.8% of airborne particles of size more than 0.2 microns and can capture dust, pollen, mold spores, bacteria, and viruses. The filter 120 can be adapted to detect real-time air quality levels (PM2.5, VOCs, CO2, and more) and may automatically adjusts the purification intensity based on air quality.

A control button 122 is disposed on the shell 102 and is preferably positioned below the grid 116. The control button 122 is adapted to activate and deactivate the system 100 manually. The control button 122 can be a physical button or can also be a touch button. In one embodiment, the control button 122 is biometric enabled for enabling authenticated access to the system 100. A base 124 extends downwardly from the bottom end 106 of the shell 102 and is adapted to create and maintain a stable electrical connection with a socket such as a light socket in a wall/ceiling, a vehicle lighter socket, or in a light or lamp fixture. The base 124 can be any screw base including but not limited to E26, E27, E12, E14, E17, and E39.

The shell 102 is preferably insulating and lightweight and further can come in different colors and designs. Preferably, the top end 114 of the fan unit 108 has a smaller circumference than the circumference of the top end 104 of the shell 102 for providing an aesthetic appearance and a curved orientation to the fan inlets 110 for better absorption of surrounding air. It will be apparent to a person skilled in the art that the air purifying system 100 resembles like a light bulb and can be used easily without requiring any professional help. In some embodiments, the device 100 may include separate control buttons for illumination source 112 and the fan unit 108, thereby enabling independent operation thereof.

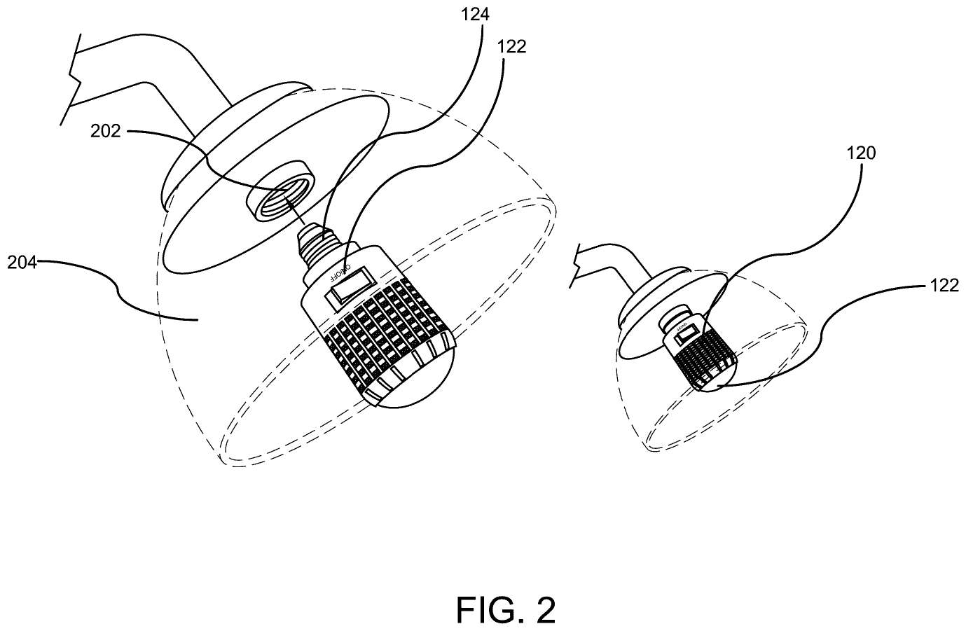

FIG. 2 illustrates a perspective view showing the combined lamp and air purifier system of the present invention being screwed into a light bulb threading on a lamp in accordance with the disclosed structure. The air purifying system 100 can be operatively connected to any light socket. As illustrated, the base 124 of the system 100 is screwed to the light bulb threading 202 of a light or lamp fixture 204 such that the air purifying lamp system 100 is secured and when the control button 122 is used for activating the system 100, the fan unit inlets 110 receive air which is purified by the filter 120 and is expelled through the apertures 118. The illumination source 112 is simultaneously activated to provide illumination in the surrounding area. The air purifying lamp system 100 can be unscrewed from the threading 202 when is not required or when is to be replaced with a new unit.

FIG. 3 illustrates an exploded view of the air purifying lamp system of the present invention in accordance with the disclosed structure. As illustrated, an illumination chip 302 is included in the system 100 which provides illumination and the diffuser 304 together with the illumination chip 302 forms the illumination source 112. A fan 306 is included in the shell 102 and is adapted to pull air through the inlets 110 disposed in the fan unit 108. A controller 308 controls the operation of the fan 306 and the chip 302 upon receiving an instruction from the control button 122. The controller 308 is operatively coupled to the control button 122 and controls operation of the system 100 in real time. A wireless transceiver 310 is included in the system 100 and enables a user to remotely control (activate and deactivate) the system 100.

The fan unit 108 and the shell 102 are detachably attached and preferably, when the fan unit 108 is removed, the illumination source 112 remains operatively connected to the shell 102. The shell 102 also functions as a heat sink for the system 100.

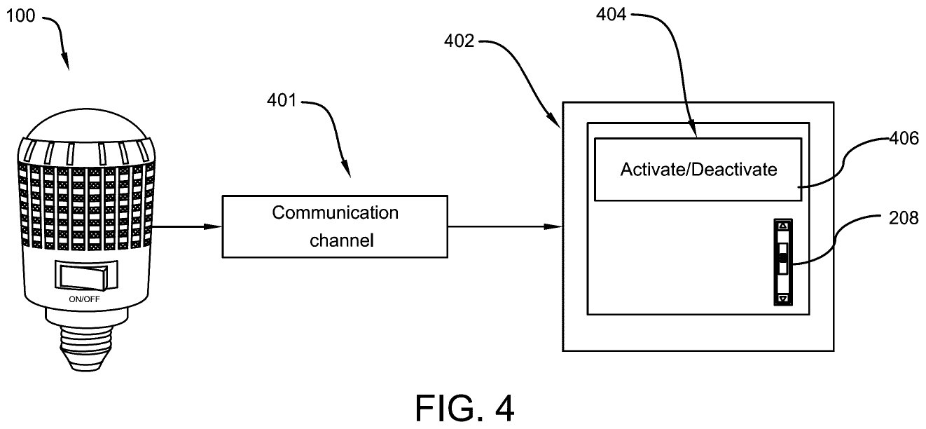

FIG. 4 illustrates a perspective view showing communication of a remote device with the air purifying system of the present invention in accordance with the disclosed structure. As illustrated, the air purifying system 100 can be wirelessly coupled to a remote device 402 such as a handheld computing device which includes a software application 404. The coupling can be done using any conventional wireless communication channel 401 and preferably using Bluetooth. The software application 404 includes an activate/deactivate option 406 for operating the system 100. Further, the application 404 includes a color change scroll bar 408 for enabling a user to change color of the illumination source 112.

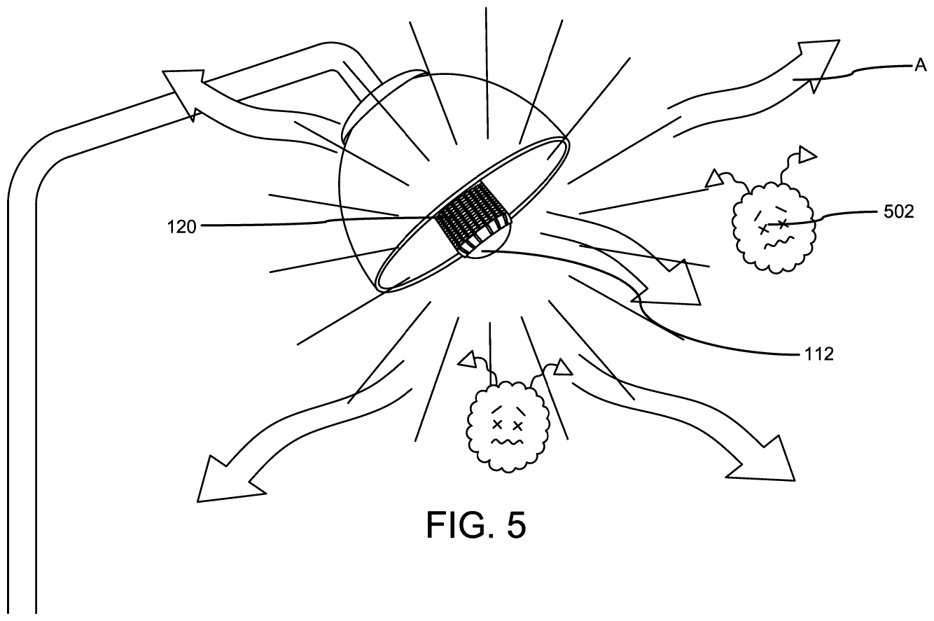

FIG. 5 illustrates a perspective view showing the air purifier system purifying air and eliminating pathogens in accordance with one embodiment of the present invention. The system 100 in the preferred embodiment performs dual filtering of air by using UV lamp 112 to kill pathogens 502 through illumination and the air filter 120 filtering air. The filtered air is transmitted back to the surroundings as illustrated by Arrow A. The system 100 provides purified air and illumination in a 360-degree direction or orientation and can be used in both residential and business settings.

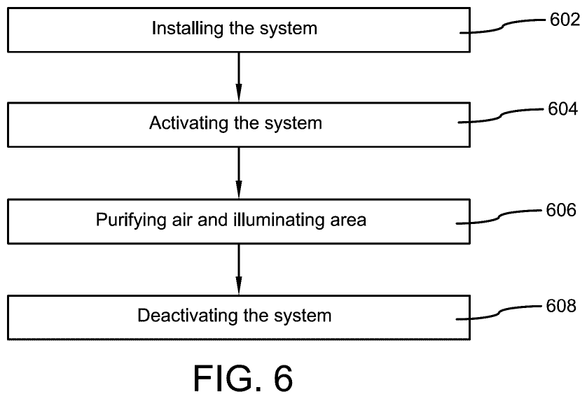

FIG. 6 illustrates a flow chart depicting a process of use of the air purifying system of the present invention in accordance with the disclosed structure. Initially, the system 100 is installed at an appropriate location and preferably by threading the base 124 to a socket (Step 602). Then, the control button 122 or the remote device 402 is used for activating the system 100 (Step 604). Thereafter, the air is purified by the system 100 while simultaneously providing illumination in the surrounding area (Step 606). Finally, after use of the system 100, the system 100 is deactivated to save electrical power (Step 608).

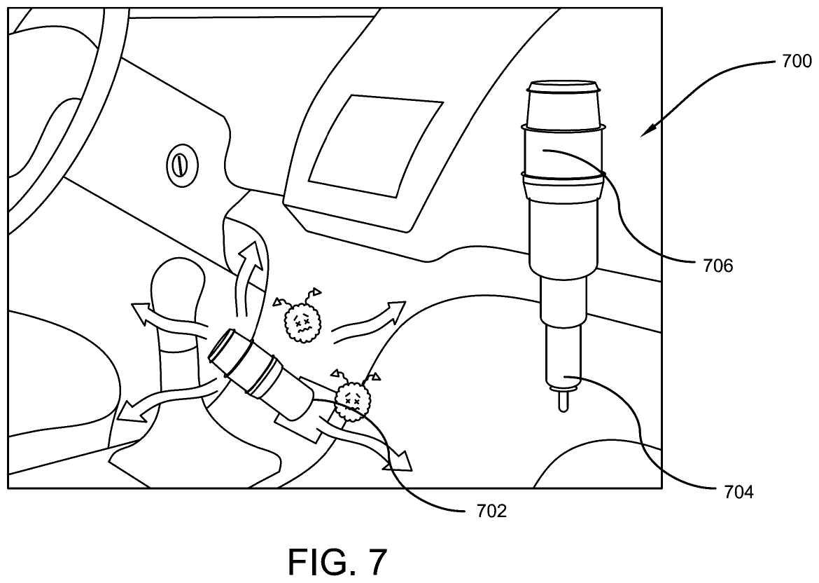

FIG. 7 illustrates a perspective view showing another embodiment of the air purifier lamp system of the present invention in accordance with the disclosed structure. In the present embodiment, the air purifier illumination device 700 is designed to be coupled to a vehicle's cigarette lighter port 702. The base 704 of the device 700 is compatible to a conventional cigarette lighter port 702. The device 700 may include an air freshener module 706 for enabling purified and fragrant air in the vehicle.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “air purifier illumination device”, “combined lamp and air purifier system”, “air purifying system”, “air purifying lamp system”, and “system” are interchangeable and refer to the combined air purifier and illumination device 100, 700 of the present invention.

Notwithstanding the forgoing the combined air purifier and illumination device 100, 700 of the present invention can be of any suitable size and configuration as is known in the art without affecting the overall concept of the invention, provided that it accomplishes the above stated objectives. One of ordinary skill in the art will appreciate that the combined air purifier and illumination device 100, 700 as shown in the FIGS. are for illustrative purposes only, and that many other sizes and shapes of the combined air purifier and illumination device 100, 700 are well within the scope of the present disclosure. Although the dimensions the combined air purifier and illumination device 100, 700 are important design parameters for user convenience, the combined air purifier and illumination device 100, 700 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A combined lamp and air purifier system comprising:
an air purifier device;
a shell having a top end and a bottom end;
an air filter;
a lamp;
a fan unit; and
an electrical connection base;
wherein said fan unit having a plurality of inlets;
wherein said fan unit is in threaded connection with said top end of said shell;
wherein said fan unit pulls air from a surrounding area in which said air purifier system is mounted;
wherein said lamp is a UV lamp;
wherein said air filter housed inside said shell;
wherein said shell having a grid including a plurality of apertures disposed in said shell;
wherein said electrical connection base selectively securable to an electrical socket; and
further wherein said fan unit receives air from a plurality of fan inlets, filters the air through said air filter, and expels said air through said plurality of apertures of said shell.

2. The combined lamp and air purifier system of claim 1, wherein said grid forming an area from 50% to 80% of a total area of said shell.

3. The combined lamp and air purifier system of claim 1, wherein said grid and said shell are cylindrical.

4. The combined lamp and air purifier system of claim 1, wherein said air filter extends around said grid for providing filtered air in a 360-degree area.

5. The combined lamp and air purifier system of claim 4, wherein said air filter is selected from the group consisting of a HEPA filter, an activated carbon filter, an ionic filter, and an electrostatic precipitator filter.

6. The combined lamp and air purifier system of claim 5, wherein said air purifier device having a control button for activating and deactivating said air purifier device.

7. The combined lamp and air purifier system of claim 6, wherein said electrical connection base extending downwardly from said bottom end of said shell.

8. The combined lamp and air purifier system of claim 7, wherein said electrical socket selected from the group consisting of a light socket, a vehicle lighter socket, and a lamp fixture.

9. The combined lamp and air purifier system of claim 8, wherein said UV lamp is an LED.

10. The combined lamp and air purifier system of claim 9, wherein said UV lamp is convex and extends above a top end of said fan unit.

11. The combined lamp and air purifier system of claim 9, wherein said UV lamp is operatively connected to said top end of said fan unit.

12. The combined lamp and air purifier system of claim 9, wherein said UV lamp emitting UV-C in a wavelength range of 200-800 nm.

13. The combined lamp and air purifier system of claim 9, wherein said UV lamp emitting UV-C in a wavelength range of 250-300 nm.

14. The combined lamp and air purifier system of claim 9, wherein said control button selectively actuating said fan unit and said lamp.

15. A combined lamp and air purifier system comprising:
an air purifier device;
a shell having a top end and a bottom end;
an air filter;
a lamp;
a fan unit;
an electrical connection base; and
a remote device;
wherein said fan unit having a plurality of inlets;
wherein said fan unit is in threaded connection with said top end of said shell;
wherein said fan unit pulls air from a surrounding area in which said air purifier system is mounted;
wherein said lamp is a UV lamp;
wherein said air filter housed inside said shell;
wherein said shell having a grid including a plurality of apertures disposed in said shell;
wherein said electrical connection base selectively securable to an electrical socket;
wherein said air purifier device having a control button for activating and deactivating said air purifier device;
wherein said remote device remotely controlling said control button; and
further wherein said fan unit receives air from a plurality of fan inlets, filters the air through said air filter, and expels said air through said plurality of apertures of said shell.

16. The combined lamp and air purifier system of claim 15, wherein said grid forming an area from 50% to 80% of a total area of said shell.

17. The combined lamp and air purifier system of claim 15, wherein said grid and said shell are cylindrical.

18. The combined lamp and air purifier system of claim 15, wherein said air filter extends around said grid for providing filtered air in a 360-degree area.

19. The combined lamp and air purifier system of claim 15, wherein said air filter is selected from the group consisting of a HEPA filter, an activated carbon filter, an ionic filter, and an electrostatic precipitator filter.

20. A combined lamp and air purifier system comprising:
an air purifier device;
a shell having a top end and a bottom end;
an air filter;
a lamp;
a fan unit; and
an electrical connection base;
wherein said fan unit having a plurality of inlets;
wherein said fan unit is in threaded connection with said top end of said shell;
wherein said fan unit pulls air from a surrounding area in which said air purifier system is mounted;
wherein said lamp is a UV lamp;
wherein said air filter housed inside said shell;
wherein said shell having a grid including a plurality of apertures disposed in said shell;
wherein said electrical connection base selectively securable to an electrical socket;
wherein said fan unit receives air from a plurality of fan inlets, filters the air through said air filter, and expels said air through said plurality of apertures of said shell;
wherein said electrical socket selected from the group consisting of a light socket, a vehicle lighter socket, and a lamp fixture; and
further wherein said UV lamp emitting UV-C in a wavelength range of 200-800 nm.
